(19) United States
(12) Patent Application Publication
(10) Pub. No.: US 2008/0228095 A1
(43) Pub. Date: Sep. 18, 2008

(54) MEDICAL DEVICE

(76) Inventor: Mary RICHARDSON, Baton Rouge, LA (US)

Correspondence Address: GARVEY SMITH NEHRBASS & NORTH, LLC, LAKEWAY 3, SUITE 3290, 3838 NORTH CAUSEWAY BLVD., METAIRIE, LA, 70002, US

(21) Appl. No.: 11/686,563

(22) Filed: Mar. 15, 2007

(51) Int. Cl. A61B 5/02

(52) U.S. Cl. 600/528

(57) ABSTRACT

A multi-functional, hand-held medical device for measuring bodily functions and physiological parameters and for medical screening and diagnosis by dual sound detection. A multi-functional, hand-held medical device capable of accurate, automatic and instantaneous readings of data received from the patient's different bodily functions, enhances productivity of the user, allows for flexibility to adjust the distance between the patient and the user, and reduces the potential for transmission of infectious or contagious organisms between the patient and the user. A multi-functional, hand-held medical device that does not require the use of earpieces. A method for measuring bodily functions and physiological parameters and for medical screening and diagnosis by dual sound detection. An undoubtable useful tool not only for professionals but also for lay persons who may require frequent monitoring of vital sounds during home care assessments

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

1. Field of the Invention

The apparatus of the present invention relates generally to medical instruments for measuring bodily functions and physiological parameters, and more particularly, to an apparatus for medical screening and diagnosis utilizing dual sound detection, and more particularly, to a combined unitary stethoscope and ultrasound device.

2. General Background of the Invention

A stethoscope is an acoustic medical device for listening to internal sounds in the human body. A stethoscope is most often used to listen to heart sounds, to breathing (breath sounds) and other lung sounds, to intestinal tract sounds and to blood flow in the arteries and the veins. A stethoscope may be used for measuring blood pressure. A stethoscope may be used to aid the diagnosis of certain diseases and conditions. A stethoscope may transmit certain sounds and exclude others.

In general, there are two types of stethoscopes, acoustic and electronic. Acoustic stethoscopes are familiar to most people, and operate on the transmission of sound from the chestpiece, via air-filled hollow tubes, to the listener's ears. The chestpiece usually consists of two sides that can be placed against the patient for sensing sound, on one side is a diaphragm (usually a plastic disc) and on the other side is a bell (usually a hollow cup). The diaphragm of the stethoscope is the flat part at the end of the tubing, with the thin plastic “drum-like” covering. Some stethoscopes have a diaphragm but no bell. The bell of the stethoscope is the cup shaped part at the end of the tubing, usually opposite to the diaphragm. Not all stethoscopes have a bell. If the diaphragm is placed on the patient, body sounds vibrate the diaphragm, creating acoustic pressure waves which travel up the tubing to the listener's ears. If the bell is placed on the patient, the vibrations of the skin directly produce acoustic pressure waves traveling up to the listener's ears. The bell transmits low frequency sounds, while the diaphragm transmits higher frequency sounds. A problem with the acoustic stethoscope is that the sound level is extremely low, making diagnosis difficult.

Electronic stethoscopes overcome the low sound levels by amplifying body sounds. Electronic stethoscopes require conversion of acoustic sound waves to electrical signals which can then be amplified and processed for optimal listening. Unlike acoustic stethoscopes, which are all based on the same physics, transducers in electronic stethoscopes vary widely. Several examples are (1) placing a microphone in the chestpiece; (2) placing a piezoelectric crystal at the head of a metal shaft, the bottom of the shaft making contact with a diaphragm; (3) placing a piezoelectric crystal in foam behind a thick rubber-like diaphragm; and (4) forming a capacitive sensor in a diaphragm with an electrically-conductive inner surface so that sound waves create changes in the electric field.

FIG. 1 shows a prior art stethoscope. In general, stethoscope 1 has several parts: chestpiece 6, tubing 4 and earpieces 5. Chestpiece 6 generally consists of bell 2, and diaphragm 3. Bell 2 of stethoscope 1 is the cup shaped part at the end of tubing 4, usually opposite to diaphragm 3. Bell 2 is used to listen to low pitch sounds. Diaphragm 3 of stethoscope 1 is the flat part at the end of tubing 4, with the thin plastic “drum-like” covering. Diaphragm 3 is used to listen to high pitch sounds. In some stethoscopes 1, chestpiece 6 has diaphragm 3 but no bell 2; in some stethoscopes 1, chestpiece 6 has bell 2 but no diaphragm 3.

Tubing 4 of stethoscope 1 transmits sound from bell 2 and/or diaphragm 3 of chestpiece 6 to earpieces 5. In some stethoscopes 1, tubing 4 is a single tube; in some stethoscopes 1, tubing 4 has double tubes. Earpieces 5 prevent outside sounds from interfering with listening to the sounds picked up by bell 2 and/or diaphragm 3 of chestpiece 6.

The following U.S. Patents and Published Applications are incorporated herein by reference: U.S. Pat. Nos. 4,515,164; 4,413,629; 5,960,089; and 6,210,344; U.S. Published Patent Application Nos: 2002/0071570; 2005/0119584; 2005/0165310; and 2005/0234339.

U.S. Pat. No. 5,960,089 discloses a Doppler system in combination with a stethoscope (see column 4, line 66 to column 5, line 55). U.S. Pat. No. 4,515,164 discloses a portable Doppler device suspended from the neck that gathers medical data (see column 2, lines 48-58 and column 4, lines 8-24). U.S. Pat. No. 4,413,629 discloses a hand held portable Doppler device for gathering data normally gathered by a stethoscope (see column 3, line 60 to column 4, line 5). U.S. Publication Nos. 2005/0119584 (see paragraph 0066), 2005/0234339 (see paragraph 0005) and 2005/0165310 (see paragraph 0009) are directed to Doppler stethoscopes for gathering medical data.

### BRIEF SUMMARY OF THE INVENTION

The present invention provides a medical instrument or device for measuring bodily functions and physiological parameters and for medical screening and diagnosis by dual sound detection. The present invention provides a medical instrument capable of accurate, automatic and instantaneous readings of data received from the patient's different bodily functions, enhances productivity of the user, allows for flexibility to adjust the distance between the patient and the user, and reduces the potential for transmission of infectious or contagious organisms between the patient and the user.

The present invention provides a multi-functional, hand-held combined stethoscope and ultrasound medical device that does not require the use of earpieces. The present invention is a dual functioning medical instrument that provides ultrasound or Doppler measurement as well as acoustic, electronically amplified acoustic, or electronic sound transmission.

The present invention combines the dual capabilities of a stethoscope and ultrasound or Doppler function into an all-in-one device which condenses the labor of carrying around or looking for two separate instruments. The results provided by the present invention are automatic and obtainable audio and/or visual outputs. The present invention is small enough for individual use and is easy to use.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is perspective view of a prior art stethoscope.

FIG. 2 is a perspective view of one embodiment of the present invention.

FIG. 3 is a perspective view of another embodiment of the present invention.

FIG. 4 depects the steps of one embodiment of a method of the present invention.

### DETAILED DESCRIPTION OF THE INVENTION

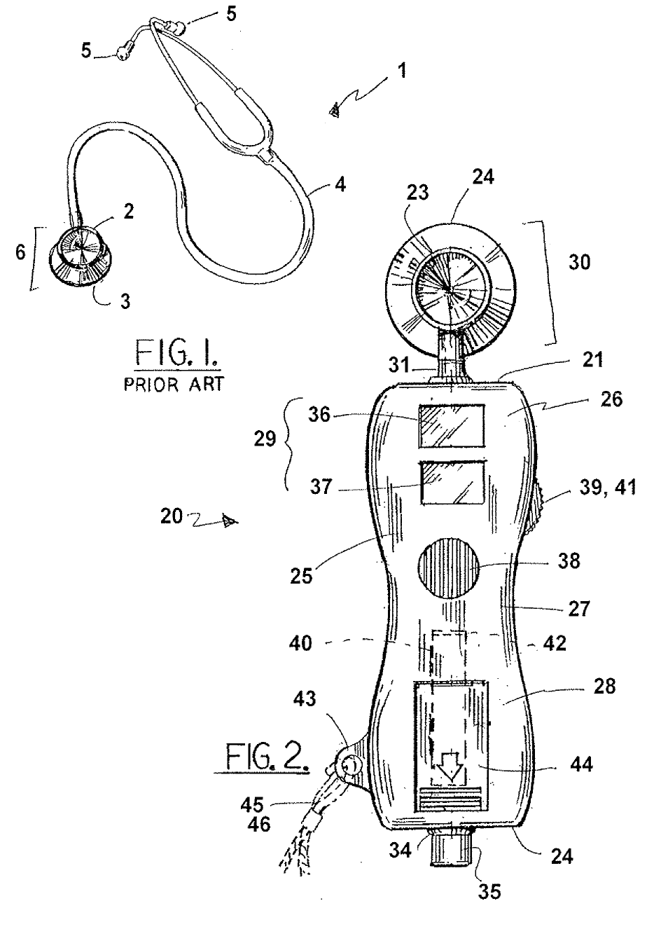

Referring now to one embodiment of the present invention, as shown in FIG. 2, medical device 20 comprises hand-held housing 25, which is comprised of upper body section 26, middle body section 27, and lowerbody section 28, chestpiece 30 and ultrasound probe or transducer 35. Like a conventional stethoscope, the present invention maybe used to monitor respiration, hypoactive or hyperactive bowel sounds, pulse beat on a minute hand clock. The present invention may be placed directly on the chest, on the abdomen, or on the extremities (legs, arms, hands, etc.), for example, to transmit sounds for measuring bodily functions and physiological parameters.

Housing 25 is formed so as to comfortably be held in the hand of the user. In one embodiment, housing 25 is shaped such that middle body section 27 is smaller than both upper body section 26 and lower body section 28. Housing 25 is preferably hand-held, and thus is preferably shaped for such of use.

When using conventional stethoscopes, generally, the user must get close to the patient due to the limited length of the stethoscope tubing. The present invention reduces the chance of transmitting infectious and/or contagious organisms because it is hand-held, allowing the user to maintain an appropriate distance from the patient. While close contact with a patient is inevitable, the present invention allows the user to maintain a greater distance than that achieved when using a conventional stethoscope.

Chestpiece 30 is preferably located at extreme end 21 of upper body section 26. However, chestpiece 30 may be located anywhere on housing 25, so long as its function is not impaired. Chestpiece 30 may be connected to housing 25 by neck 31. Neck 31 allows chestpiece 30 to be fully rotatable. Rotating of chestpiece 30 assists the user in examination of both adult and pediatric patients.

Chestpiece 30 functions to observe acoustic sounds from the patient, as is known in the art and described in U.S. Pat. No. 3,951,230 which disclosure is incorporated herein by reference. Chestpiece 30 may be bell 2 or diaphragm 3, as is well known in the art.

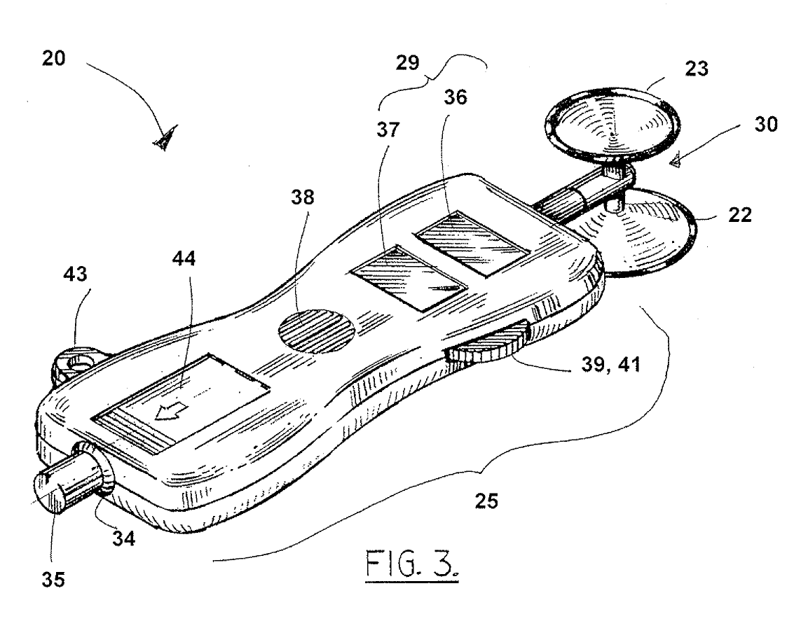

In the present invention, chestpiece 30 is dual sided 22, 23 such that it may be used with both adult patients and with pediatric patients. As shown in FIGS. 2 and 3, chestpiece 30 has pediatric side 23 attached on the reverse of adult side 22. Sides 22, 23 are appropriately sized for the type of patient, i.e. pediatric side 23 is sized smaller than adult side 22. Pediatric side 23 also may be used on petite adults. Conventional stethoscopes are sized for either adult or for pediatric patients, not both. Due to the dual sides 22, 23 of chestpiece 30, chestpiece 30 has dual function.

In one embodiment, chestpiece 30 and probe or transducer 35 are located at opposing ends of housing 25. Extreme end 24 of lower body section 28 may provide insert 34 for probe 35. If probe 35 is connected by insert 34, then probe 35 may be removable. Probe 35 would be removable when a gel should be applied to areas where the pulse is not easily detected, such as the wrist or foot. In an alternative embodiment, probe 35 maybe integrally connected to housing 25 at either upper body section 26 or lower body section 28.

Probe or transducer 35 functions using ultrasound, or Doppler, sound detection as is known in the art and is described in U.S. Pat. No. 4,413,629 which disclosure is incorporated herein by reference. The Doppler effect is a change in the frequency of sound waves caused by moving objects. Ultrasound uses sound waves to generate information, for example, when a sound wave strikes an object, it bounces backward, or echoes. By measuring these echo waves it is possible to determine how far away the object is and its size, its shape, its consistency (whether the object is solid, filled with fluid, or both) and its uniformity. Probe or transducer 35 both generates and receives high frequency, inaudible, sound waves and records the echoing waves. Probe 35 is pressed against a patient's skin, or other body part being examined. Generally, a gel is applied to the skin and then probe 35 is moved over the body part being examined. As probe 35 slides over a patient's skin, it conducts transmitting sound waves that are reflected or refracted back to probe 35 by the patient's internal organs, tissues, bones and body fluids. As the sound waves bounce off of internal organs, tissues bones and body fluids, probe 35 records tiny changes in the sound's pitch and direction. These signature waves are instantly measured so that the specific characteristics of the object are obtainable. Probe 35 then sends this information to electronics package 42, which transmits the sound waves to audible patterns for speaker 38. Probe 35 may be utilized to detect sound in any pulseless extremity such as a patient's arm or foot, or to listen for the sound of blood flow through a patient's blood vessels.

Located on housing 25 is view screen 29. View screen 29 provides a display of the bodily functions being observed, for example, view screen 29 may display the pulse rate, the heartbeat data, or the respiration rate data of the patient. View screen 29 may also display additional information as necessary. Multiple view screens 36, 37 may be provided as desired. In one embodiment, view screen 29 is located on upper body section 26 and comprises first view screen 36 and second view screen 37. First view screen 36 displays the pulse and/or heartbeat data and secondview screen 37 displays respiration data. View screen 29, 36 and/or 37 may be illuminated, as is generally known, such as by LED or other lighting.

Speaker 38 is provided on housing 25 to produce the sounds received by chestpiece 30 or probe 35. In one embodiment, speaker 38 is located on upper body section 26 near middle body section 27; however, speaker 38 may be located on elsewhere on housing 25, so long as its function is not impaired. The present invention eliminates the ear discomfort users of conventional stethoscopes experience. Speaker 38 of the present invention removes the necessity of earpieces to hear the sounds emanating from the patient.

Volume control 39 is located on housing 25. In one embodiment, volume control 39 is located on upper body section 26 near middle body section 27 so as to make adjustment of volume easy when device 20 is being held. Volume control 39 allows the user to adjust the volume of sounds transmitted from chestpiece 30 and/or probe 35. The volume may be adjusted to an appropriate level to allow the user to adequately hear the sounds received from the heart, lungs, bowels, etc.

The present invention requires power source 40. Power source 40 may be conventional battery, rechargeable battery or AC operated. In one embodiment, power source 40 is located in the interior of lower body section 28. Power source 40 may additional include an AC connection for direct power from an electrical outlet or to recharge a rechargeable battery maintained within device 20, as is generally known. Power source 40 may also provide illumination for view screen 29, 36 and/or 37

Power switch 41 allows for turning the present invention on or off. In one embodiment, power switch also may function as volume control 39, as is generally known. Power switch should be off when device 20 is not in use to conserve power.

Housing 25 contains the necessary electronics or mechanisms 42 for operating device 20 as described, for example, for transmitting sound picked up by chestpiece 30 and/or probe 35 to speaker 38 and to adjust the sounds emanating from speaker 38 through volume control 39, as is generally known. Housing 25 also contains the necessary electronics 42 for transforming the sound picked up by chestpiece 30 and/or probe 35 to readable form for display on view screen 29, 36, 37, and for illumination of view screen 29, 36, 37, as is generally known. Housing 25 further contains the necessary mechanisms 42 to utilize power from power source 40 and to allow the function of power switch 41 for turning device 20 on or off and to provide power to the necessary electronics, as is generally known.

In an alternative embodiment, device 20 has carrying means 43 whereby the user may carry device 20 without the use of hands, for example, pocket clip 44, key ring 45 or garment strap 46. See, for example, U.S. Pat. No. 6,279,362, which disclosure is incorporated herein by reference. Carrying means 43 alleviates neck stress and back pain that a user sometimes experiences when having to hang a conventional stethoscope from the neck. Carrying means 43 may improve the user's posture. Carrying means 43 may lessen the likelihood of device 20 of being misplaced as it no longer needs to be put down as often.

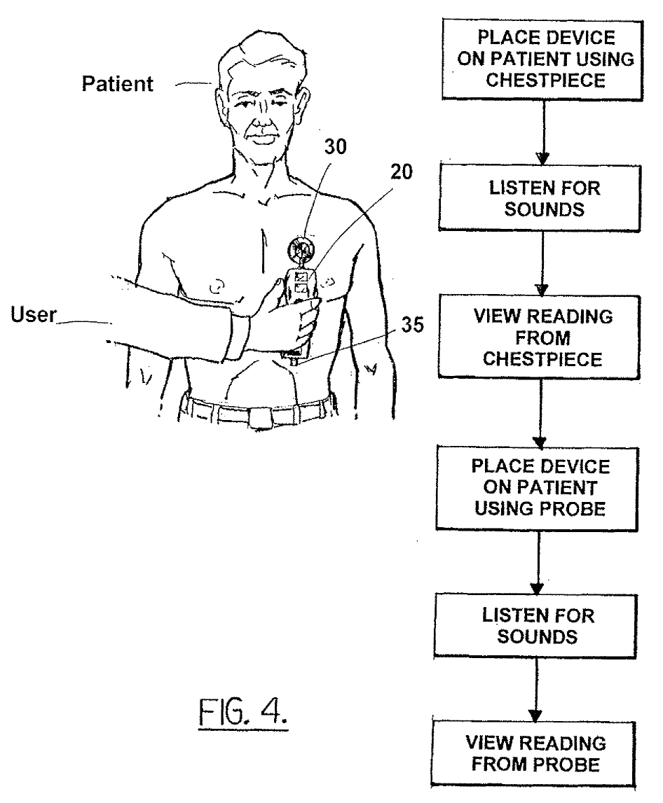

In another embodiment of the present invention as shown in FIG. 4, a user turns device 20 on using power switch 41. Chestpiece 30 of device 20 is then placed over the preferred listening body part or system of a patient, for example, chest, abdomen or limb. User then listens for sounds transmitted from chestpiece 30 through speaker 38. User may adjust volume from speaker 38 by using volume control 39. User notes the heartbeat/pulse or respiration number reading from view screens 36, 37. If sound is not easily detected using chestpiece 30, then user may repeat previous steps using probe 35.

| Parts Number | Description |
|---|---|
| 1 | prior art stethoscope |
| 2 | bell |
| 3 | diaphragm |
| 4 | tubing |
| 5 | earpieces |
| 6 | chestpiece |
| 20 | medical device |
| 21 | end (upper body section) |
| 22 | side (chestpiece) |
| 23 | side (chestpiece) |
| 24 | end (lower body section) |
| 25 | hand-held housing |
| 26 | upper body section |
| 27 | middle body section |
| 28 | lower body section |
| 29 | view screen |
| 30 | chestpiece |
| 31 | neck |
| 34 | insert |
| 35 | ultrasound/Doppler probe or transducer |
| 36 | first view screen |
| 37 | second view screen |
| 38 | speaker |
| 39 | volume control |
| 40 | power source (battery compartment) |
| 41 | power switch |
| 42 | electronics or mechanisms |
| 43 | carrying means |
| 44 | pocket clip |
| 45 | key ring |
| 46 | garment strap |

The foregoing embodiments are presented by way of example only; the scope of the present invention is to be limited only by the following claims.

1. A medical device comprising:
a) a hand-held housing having a gripping surface that enables a user to firmly grasp the device;
b) a chestpiece for refined acoustic sound detection connected to the housing;
c) a speaker on the housing;
d) a volume control on the housing for controlling the level of sound output by the speaker;
e) an ultrasound probe connected to the housing;
f) a first view screen on the housing for providing visual interpretation of sounds detected by the chestpiece or the probe;
g) a first electronics package for interfacing the chestpiece, the volume control, the speaker and the view screen;
h) a second electronics package for interfacing the ultrasound probe, the volume control, the speaker and the view screen.

2. A device according to claim 1, wherein the chestpiece is rotatably connected to the housing.

3. A device according to claim 1, wherein the chestpiece is dual sided.

4. A device according to claim 1, wherein the probe is removable.

5. A device according to claim 1, wherein the chestpiece and the probe are located at opposing ends of the housing.

6. A device according to claim 1, further comprising a second view screen on the housing.

7. A device according to claim 6, wherein one of the first view screen and the second view screen displays pulse data and wherein the other of the first view screen and the second view screen displays respiration data.

8. A device according to claim 1, wherein the volume control also functions as a power switch.

9. A device according to claim 1, further comprising a power switch.

10. A device according to claim 1, wherein the housing is shaped for a hand.

11. A method for measuring bodily functions and physiological parameters and for medical screening and diagnosis of a patient by dual sound detection utilizing a chestpiece and a probe, comprising the steps of:
a) placing the chestpiece on the patient;
b) listening for sounds transmitted from the chestpiece to a speaker; and
c) viewing a first reading for the sound transmitted from the chestpiece.

12. The method of claim 11, further comprising the steps of:
d) placing the probe on the patient when the sound transmitted from the chestpiece is not detected;
e) listening for sounds transmitted from the probe to the speaker; and
f) viewing a second reading for the sound transmitted from the probe.

13. A medical device as shown and described.
